(19) United States
(12) Patent Application Publication
(10) Pub. No.: US 2002/0016304 A1
(43) Pub. Date: Feb. 7, 2002

(54) CARRIER FOR STABILIZING NUCLEIC ACID

(76) Inventors: ATSUSHI MARUYAMA, YOKOHAMA-SHI (JP); TOSHIHIRO AKAIKE, HOYA-SHI (JP); TAKESHI GOTO, TSUKUBA-SHI (JP); KEISHI YONEMURA, TSUKUBA-SHI (JP)

Correspondence Address: PILLSBURY MADISON & SUTRO, INTELLECTUAL PROPERTY GROUP, 1100 NEW YORK AVENUE, NW, NINTH FLOOR, EAST TOWER, WASHINGTON, DC, 200053918

(21) Appl. No.: 09/267,608

(22) Filed: Mar. 15, 1999

Related U.S. Application Data
(63) Continuation-in-part of application No. 08/850,928, filed on May 2, 1997, now abandoned.

(30) Foreign Application Priority Data
May 16, 1996 (JP) 146825/1996
Oct. 4, 1996 (JP) 283259/1996

(51) Int. Cl. A61K 48/00; A61K 9/16; A61K 9/50; A61K 9/14; A61K 31/785

(52) U.S. Cl. 514/44; 424/78.18; 424/78.22; 424/78.17; 424/486; 424/497; 424/499; 435/320.1; 435/455

(57) ABSTRACT

A carrier for stabilizing a nucleic acid is provided which forms a soluble, stable complex with a nucleic acid, which has a stabilizing effect on a double or triple helix of a nucleic acid, which suppresses the structural change of a nucleic acid, and which enables the reversibility of a nucleic acid to be retained. A method for stabilizing a nucleic acid by use of such a carrier is also provided. This carrier is a polymer having a poly(cationic amino acid) as a main chain, and a comb-shaped hydrophilic group as a side chain modifying the polymer.

### RELATED APPLICATION

This application is a continuation-in-part of application Ser. No. 08/850928 filed on May 2, 1997, now pending.

### BACKGROUND OF THE INVENTION

1. Field of the Invention

The present invention relates to a carrier for stabilizing a nucleic acid, and a method for stabilizing a nucleic acid by using the carrier for stabilizing nucleic acid.

2. Related Background Art

With the rapid progress of genetic engineering, various molecular biological methods have been developed, resulting in the analysis of genetic information and the elucidation of gene functions. Attempts to apply the achievements to therapeutic methods have accumulated.

One of the most advanced fields is gene therapy. The causative genes for varieties of genetic diseases have been discovered and deciphered. Thus, gene therapy is progressing from the stage of basic experiments into the stage of clinical applications. In the United States of America, for example, 81 protocols for gene therapy were approved by the Recombinant DNA Advisory Committee (RAC), NIH, by June 1995. According to these protocols, clinical trials have already been performed in over 200 patients with genetic diseases, such as congenital immuno deficiency syndrome, familial hypercholesterolemia, and cystic fibrosis, and various type of cancer (Jikken Igaku, Experimental Medicine, Vol. 12, No. 3, 303-307 (1994)).

Gene therapy is a way of curing a disease on the cell level. One of the major technical challenges for its clinical use is how to introduce an exogenous gene into a target cell efficiently and safely. Methods for doing this have posed some problems. That is, gene therapy is broadly classified into augmentation gene therapy for adding a new (normal) gene while leaving the abnormal (pathogenic) gene intact, and the replacement gene therapy for replacing the abnormal gene by a normal gene. Both therapies necessitate the method of introducing a normal gene into the target cell efficiently and safely.

For instance in the early 1980 s, a physical technique such as microinjection was tried, but this technique was low in the introducing efficiency, and was incapable of stable introduction. Also there was technological limits for large scale culture.

Recombinant viruses (virus vectors) as carriers for the efficient introduction of exogenous genes into target cells have been developed to make the clinical use of gene therapy possible for the first time. Some kinds of virus vectors are known to be considered for use in gene therapy. These virus vectors, however, generally require very complicated methods of production, and the way of ensuring their safety has not been established. For example, a retrovirus vector derived from Moloney murine leukemia virus (MoMLV) currently draws the most attention as the virus vector that can be used for gene therapy. This vector utilizes the advantage of the mode of propagation of the retrovirus. The retrovirus is an enveloped RNA virus whose envelope protein binds to a receptor of a host cell, permitting entry into the cell. After entry, the single-stranded viral RNA is converted into a double-stranded DNA by reverse transcriptase, for integration into the genomic DNA of the infected cell. In order for this integration to occur, cells have to be proliferated. A major problem for practical use is, therefore, that the gene cannot be introduced into nondividing cells. Thus, genetic repair of a neuron, important in many inborn errors of metabolism, is impossible. In addition to nerve cells, hemotopoietic stem cells, hepatocytes, and myocytes, the cells to be treated with gene therapy, are normally in a stationary phase, so that the efficiency of gene transduction is low for these cells. Cells taken out of the body are subjected to a treatment for promoting cell division to increase the gene transducing efficiency. Introducing a gene into these cells in vivo, however, has been considered difficult. Recently, an adenovirus vector has attracted attention as being capable of gene introduction into non-dividing cells. With the adenovirus vector, however, an exogenous gene cannot be integrated into the genomic DNA of a target cell. Hence, the effect of gene introduction disappears in several weeks, or in several months at the longest. Consequently introduction of the gene must be repeated frequently, thereby increasing physical and mental burden on the patient, and decreasing the gene introduction efficiency because of the production of anti-adenovirus antibodies. Currently, a clinical trial is conducted which administers an adenovirus vector bronchoscopically to the lung for the treatment of cystic fibrosis, and this procedure has caused an inflammatory reaction probably associated with the immunogenicity and cytotoxicity of adenovirus particles. A herpesvirus vector is expected as a vector capable of introducing an exogenous gene into a neuron. Its development has not proceeded, however, since its cytotoxicity is strong and the genome size of the virus itself is as large as 150 kb. An HIV vector was developed as a vector enabling a specific gene to be introduced into CD4-positive T lymphocytes because of the host characteristics of the virus per se (Shimada T. et al., J. Clin. Invest., Vol. 88, 1043 (1991)). The HIV vector has its biggest drawback in that it may contain a wild strain. AS for an AAV (adeno-associated virus) vector, AAV of the wild type was found to be integrated into a particular position of the 19th chromosome. Based on this finding, attention was paid to it as a vector capable of targeting the position of gene integration. Recent studies, however, have shown that a recombinant AAV vector has lost this characteristic, and an exogenous gene is integrated into a nonspecific position of the chromosome. The AAV vector, moreover, is limited in the size of the introducible exogenous gene, and is defective in that only a gene less than 5 kb in size can be packaged in the vector.

Besides virus vectors, various artificial gene introduction systems have been tried for gene therapy. For examples, gene-lipid complexes relying on positively charged lipids have been developed as non-virus carriers for gene therapy. It has been pointed out, however, that these carriers are highly cytotoxic when used in large amounts (Bioconjugate Chem., Vol. 3, 323-327 (1992); Proc. Natl. Acad. Sci. USA, Vol. 89, No. 17, 7934-7938 (1992); J. Biol. Chem., Vol. 269, 12918-12924 (1994); Japanese Patent Laid-Open No.Hei 6-505980 (505980/94); and Japanese Patent Laid-Open No.Hei 6-507158 (507158/94). Many research reports also show attempts to deliver genes into target cells or other cells by utilizing the negative charges of nucleic acids or their derivatives, and forming their electrostatic complexes with synthetic high molecular derivatives having positive charges (Bioconjugate Chem., Vol. 3, 323-327 (1992); Proc. Natl. Acad. Sci. USA, Vol. 89, No. 17, 7934-7938 (1992); J. Biol. Chem., Vol. 269, 12918-12924 (1994); Japanese Patent Laid-Open No. Hei 6-505980 (505980/94); and Japanese Patent Laid-Open No. Hei 6-507158 (507158/94)). However, it has been indicated since the early days that the positively charged synthetic polymers, when used alone, have high cytotoxicity (Bioconjugate Chem., Vol. 1, 149-153 (1990)). At the same time, when derivatives of these synthetic polymers are administered in vivo, they are recognized as foreign bodies, thus exerting an effect on the immune system, such as an anaphylactic shock.

Attempts at using nucleoproteins from the living body have been made, because they are recognized, with difficulty, as foreign bodies. The nucleoproteins have the nature to bind specifically to nucleic acids and their derivatives. Thus, they can form electrostatic complexes with these substances, serving as vectors for introduction of genes. In some studies, histone proteins, nucleoproteins, were used as carriers of plasmid DNA (Yasufumi Kaneda et al., Science, Vol. 243, 375-378 (1989); Mirjam Breeuwer and David S. Goldfarb, Cell, Vol. 60, 999-1008 (1990); and Jian Chen et al., Human Gene Therapy, Vol. 5, 429-435 (1994)). Even these studies are merely concerned with how to incorporate genes into cells, and are not necessarily aimed at improving the efficiency of gene expression in many cell species. The nucleoproteins of these studies are questionable in terms of their applicability as vectors for introducing genes into the cytoplasm efficiently in many cell species.

A DNA triple helix formed by Watson-Crick double helix present in a cell and a third exogenous gene added thereto can control the expression of a particular gene by the exogenous gene, and can also permanent inactivation of the gene.

In the Watson-Crick double helix, the double-stranded helical structure is known to be stabilized by hydrogen bonds between guanine (G) and cytosine (C) and between adenine (A) and thymine (T). The triple helix is known to involve the third strand binding to the double helix, like TAT, by Hoogsteen type hydrogen bonding.

As an application of triple-stranded DNA formation involving the exogenous gene linked to the double-stranded DNA in the cell, it has been known that the exogenous gene is modified with a molecule which will chelate metal ions (e.g., EDTA or phenanthroline), and the double-stranded DNA can be cleaved by damaging it with the modified gene (Pete E. Nielsen, Bioconjugate Chem., Vol. 2, No. 1, 1-12 (1991)). It is also possible to modify the exogenous gene with a crosslinking agent, thereby to achieve the permanent control of expression of a particular intracellular gene. The specific cleavage of DNA in the cell by use of triple-stranded DNA formation also enables applications to fields dealing with giant DNA, such as a genome project. In terms of controlling gene expression, this technology has attracted increasing attention as applicable to the treatment of AIDS (W. Michael McShan et al., Biol. Chem., Vol. 267, No. 8, 5712-5721 (1992)) or the treatment of cancer. Actually, the polypurine or polypyrimidine sequence of the human cancer gene c-myc was noticed, and a polypurine chain of nucleotide corresponding to this region was added from the outside. As a result, a triple-stranded structure was formed, successfully suppressing the expression of c-myc in vitro and then in vivo (E. H. Postel, S. J. Flint, D. J. Kessler and M. E. Hogan, Proc. Natl. Acad. Sci. USA, Vol. 88, 8227-8231 (1991)).

However, the triple-stranded DNA is very unstable under physiological conditions, and its formation is difficult, because of electrostatic repulsion between anions of the DNA s.

To solve these problems, it is essential to develop a transporter for transporting an exogenous gene to a targeted place, and to develop a stabilizer for promoting the formation of a triple helix and stabilizing the resulting triple helix. Hitherto known as stabilizing agents for DNA double helix formation and DNA triple helix formation are low molecular polyamines and intercalating agents. Low molecular polyamines, such as spermine and spermidine, are known to show a stabilizing effect by a mechanism by which protonated polyamines suppress the repulsion of the ions of DNA s (Thresia Thomas and T. J. Thomas, Biochemistry, Vol. 32, No. 50, 14068-14074 (1993); and Ching-Hsuan Tung, Kenneth J. Breslauer and Stanley Stein, Nucleic Acids Research, Vol. 21, No. 23, 5489-5494 (1993)). In fact, however, these polyamines are replaced by other ions under physiological conditions, showing a low stabilizing effect. Acridine and psoralen are known to intercalate between base pairs, thereby resulting in stabilization (Mikhail Grigoriev et al., Proc. Natl. Acad. Sci. USA, Vol. 90, 3501-3505 (1993)). Psoralen can bind to a double helix upon photo-crosslinking. Benzo[e]pyridoindole derivatives are known to have both of the features of intercalating agents and polyamines and stabilize a triple helix in a specific manner (J. L. Mergny et al., Science, Vol. 256, 1681-1684 (1992)). However, these intercalating agents generally involve problems such that they stabilize a double helix mainly, but their stabilizing effect is weak for a triple helix, and they may be toxic or carcinogenic.

Polylysine, a high molecular polycation, is known to firmly bind to DNA, and relieve ion repulsion between the phosphate groups of the DNA, thereby bringing stabilization. When a complex is formed from polylysine alone and DNA, however, compaction or precipitation occurs, so that a particular DNA cannot be recognized, nor can a double helix or triple helix be formed.

### SUMMARY OF THE INVENTION

We, the inventors of the present invention, have conducted in-depth studies in the light of the following problems with the earlier technologies:

(1) Conventional intercalating agents, such as protonated polyvalent polyamines, acridine and psoralen, can stabilize double-stranded DNA formation, but are not very effective in stabilizing triple-stranded DNA formation under physiological conditions.

(2) Polylysine, a high molecular polycation, firmly binds to DNA to be introduced into a cell, causing compaction or precipitation in the DNA. When the DNA is introduced in the cell, therefore, a particular DNA cannot be recognized any longer, nor can a double helix or triple helix be formed. Based on these studies, we have succeeded in finding a novel carrier for stabilizing nucleic acid that has solved the above problems. This success has led us to accomplish the present invention.

That is, an object of the present invention is to obtain

(1) a carrier which easily solubilizes a complex of a gene to be introduced into a cell with carrier;

(2) a carrier which suppresses the structural change of an exogenous gene and which has the excellent function to retain the reversibility of the exogenous gene, and

(3) a carrier for stabilizing a nucleic acid, the carrier having the effect of stabilizing a DNA double helix or a DNA triple helix formed by introducing an exogenous gene into a cell.

To attain the foregoing object, the present invention provides a carrier for stabilizing a nucleic acid, the carrier having a poly(cationic amino acid) as a main chain, and having as a side chain a hydrophilic group chemically bound to the poly(cationic amino acid) in a combed shape.

The present invention also provides a carrier for stabilizing a nucleic acid, in which the poly(cationic amino acid) is polylysine.

The present invention further provides a carrier for stabilizing a nucleic acid, in which the hydrophilic group is at least one member selected from the group consisting of polyethylene glycol(PEG), and polyethylene glycol derivatives, and saccharides.

The present invention also provides a carrier for stabilizing a nucleic acid, in which the hydrophilic group is at least one member selected from the group consisting of glycosaminoglycan, dextran and polyethylene glycol (PEG), and polyethylene glycol derivatives.

The present invention also provides a carrier for stabilizing a nucleic acid, in which the poly(cationic amino acid) and the hydrophilic group are graft-polymerized, and graft-copolymerized.

The present invention also provides a carrier for stabilizing a nucleic acid, in which 10 to 95% by weight of the carrier for nucleic acid stabilization is the hydrophilic group.

The present invention also provides a carrier for stabilizing a nucleic acid, in which the ratio of X to Y, (X/Y) is in the range 0.5 to 2, X being the number of the cationic amino acid groups constituting the poly(cationic amino acid), and Y being the number of the phosphate groups contained in the DNA to be formed into a complex with the carrier for nucleic acid stabilization.

The present invention further provides a method for forming a complex of the nucleic acid with a carrier for stabilizing a nucleic acid, the carrier having a poly(cationic amino acid) as a main chain, and having as a side chain a hydrophilic group chemically bound to the poly(cationic amino acid) in a combed shape.

The present invention also provides the above method in which the ratio of X to Y, (X/Y) is in the range 0.5 to 2, X being the number of the cationic amino acid groups constituting the poly(cationic amino acid), and Y being the number of the phosphate groups contained in the DNA to be formed into a complex with the carrier for nucleic acid stabilization.

The present invention will become more fully understood from the detailed description given hereinbelow and the accompanying drawings which are given by way of illustration only, and thus are not to be considered as limiting the present invention.

Further scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF DRAWINGS

FIG. 1 is a view showing the chemical formulae of polylysine, dextran, and a polylysine-dextran modified polymer (PLL-g-Dex) of the present invention;

FIG. 2A is a view schematically showing the route for synthesis of the dextran-modified polymer of the present invention;

FIG. 2B is a view showing the results of NMR measurement of poly-L-lysine-g-dextran of the present invention, in which signals by dextran are indicated by thin oblique lines, while signals by poly-L-lysine are indicated by thick oblique lines;

FIG. 3 is a view showing the results of NMR measurement of a PEG modified polymer related to the present invention;

FIG. 4 is a view showing the relationship between the charge ratio and the absorbance (solubility, turbidity) in a complex of the dextran modified polymer or polylysine with DNA;

FIG. 5A is a view showing changes in the CD spectrum of a complex of the dextran modified polymer or polylysine with DNA in 1/100 PBS in response to changes in their charge ratio;

FIG. 5B is a view showing changes in the CD spectrum of a complex of the dextran modified polymer or polylysine with DNA in PBS in response to changes in their charge ratio;

FIG. 6A is a view showing aggregation due to the formation of a complex of the dextran modified polymer relevant to the present invention or polylysine with DNA;

FIG. 6B is a view showing that the complex of the dextran modified polymer relevant to the present invention with DNA is solubilized in the presence of a hydrophilic group;

FIG. 7A is a view showing changes from double-stranded DNA to single strands and vice versa according to temperature;

FIG. 7B is a view showing changes in the absorbance (melting curve, dextran modified polymer, 0.5 mM EDTA, 150 mM NaCl, pH=7.2 sodium phosphate buffer) according to changes in temperature when only poly(dA) poly(dT) was used; and their primary differential values;

FIG. 7C is a view showing changes in the absorbance (melting curve, dextran modified polymer, 0.5 mM EDTA, 150 mM NaCl, pH=7.2 sodium phosphate buffer) according to changes in temperature when PLL/DNA=0.2; and their primary differential values;

FIG. 7D is a view showing changes in the absorbance (melting curve, dextran modified polymer, 0.5 mM EDTA, 150 mM NaCl, pH=7.2 sodium phosphate buffer) according to changes in temperature when PLL-g-Dex/DNA=2; and their primary differential values;

FIG. 8A is a view schematically showing the melting curves and melting phenomena of DNA triple helix (dextran modified polymer, 0.5 mM EDTA, 150 mM NaCl, pH=7.2 sodium phosphate buffer);

FIG. 8B is a view showing that triple-stranded DNA changes into a single strand and a double helix, and then into single strands, in two steps according to temperature in the absence of PLL-g-Dex;

FIG. 8C is a view showing that triple-stranded DNA changes into single strands according to temperature in the presence of PLL-g-Dex;

FIG. 9 is a chart showing the dextran modified polymer and spermine concentration dependency of the melting point Tm of DNA triple helix (0.5 mM EDTA, 150 mM NaCl, pH=7.2 sodium phosphate buffer);

FIG. 10 is a chart showing the NaCl concentration dependency of the melting points Tm of the dextran modified polymer complex with DNA triple helix and of DNA triple helix only (0.5 mM EDTA, pH=7.2 sodium phosphate buffer);

FIG. 11 is a view showing an electrophoretogram of a complex of the dextran modified polymer or polylysine with DNA as a test of the stability in serum:

FIG. 12A is a view showing the relationship between the charge ratio and the absorbance of a complex of the dextran modified polymer with DNA (Poly(dA), Poly(dT)) when Dex2600 was used;

FIG. 12B is a view showing the relationship between the charge ratio and the absorbance of a complex of the dextran modified polymer with DNA (Poly(dA), Poly(dT)) when Dex5900 was used;

FIG. 13 is a view showing the relationship between the charge ratio and the absorbance of a complex of the dextran modified polymer with DNA (oligonucleotide); and

FIG. 14 is a view showing the relationship between the charge ratio and the absorbance of a complex of the dextran modified polymer with DNA (plasmid).

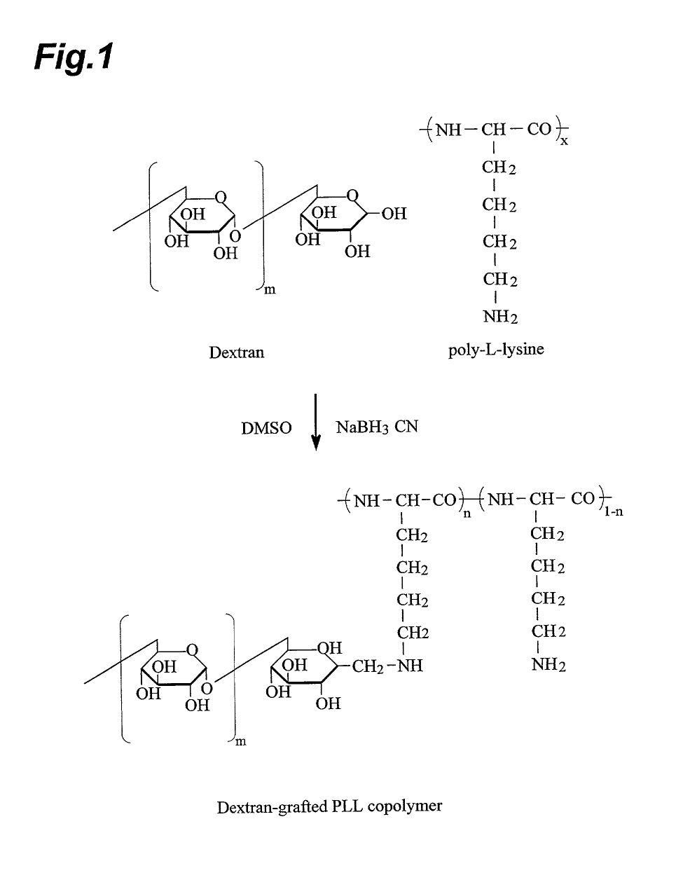

### DESCRIPTION OF THE PREFERRED EMBODIMENTS

### Carrier for Stabilizing Nucleic Acid

The carrier for stabilizing nucleic acid of the present invention has a polymer of a cationic amino acid as a main chain, and also has as a side chain bound to the main chain by chemical modification or chemical bonding. Concretely, the carrier has a poly(cationic amino acid) as a main chain, and a side chain by graft copolymerization. Examples of the poly(cationic amino acid) are polylysine, polyornithine, (ornithine-serine)copolymer, block copolymer of polylysine with polyethylene glycol (PEG), block copolymer of polyornithine with PEG, and block copolymer of poly(ornithine-serine) with PEG. Synthesis of these poly(cationic amino acids) depends on the desired degree of polymerization, but can be performed by a known ordinary method of polymerization. For instance, the polymers can be synthesized by polymerizing -carbobenzoxy-lysine-N-carboxylic acid anhydrides and benzyl-serine-N-carboxylic acid anhydrides using primary amines, such as polyethylene oxides (molecular weight 200 to 250,000) with an amino group at one terminal, as initiators. In the resulting polyethylene oxide-poly(amino acid) block copolymers, the molecular weight of the poly(amino acid) portion is not particularly limited, but preferably 200 to 50,000.

Furthermore, the carrier for stabilizing nucleic acid of the present invention has, as a side chain, a hydrophilic group incorporated into (bound to) the poly(cationic amino acid) main chain in a combed shape. The method of incorporation is not particularly limited, but graft polymerization is preferred. Examples of the hydrophilic group incorporated in a combed shape by graft copolymerization are polyethylene glycol (PEG), PEG derivatives (e.g. aldehyde derivatives of methoxy polyethyleneglycol, amino acid derivatives, or carboxymethyl derivatives), glycosaminoglycans (heparin, chondroitin sulfate), saccharides such as monosaccharides, oligosaccharides and polysaccharides (e.g. dextran, amylose), polyacrylamide, hydroxypropyl cellulose (HPC), hydroxypropyl methylcellulose (HPMC), and synthetic water-soluble polymers. Particularly preferred as the side chain hydrophilic group in the invention are dextran (Dex), and polyethylene glycol (PEG).

The method of synthesizing the graft copolymer having poly(cationic amino acid) as a main chain and a hydrophilic group as a side chain is not particularly limited, either. It can be performed by a known ordinary method of organic synthesis. That is, the side chain can be incorporated into the functional group of the main chain polymer by a suitable reaction for forming bonds. Preferably, for example, when a polysaccharide chain is to be used as a side chain, the reducing end of the saccharide chain is reacted with the amino group of the poly(amino acid) to form a bond. Particularly, a Schiff base is first formed, followed by further reduction, to form an amino bond. Alternatively, when a polysaccharide, such as dextran, is to be used as a hydrophilic side chain, the reducing end of the polysaccharide is oxidized using iodine or the like, for conversion into a carboxylic acid, followed by lactonization, to effect coupling with the amino group. An unspecified portion, rather than the terminal of the polysaccharide, may be linked to polylysine or the like. An example of the method for this synthesis is to oxidize the polysaccharide with periodic acid to form an aldehyde group, and then reductively aminate this group with the amino group of poly(lysine) or the like.

For the formation of the graft copolymer containing polyethylene glycol as the hydrophilic side chain, a Schiff base is first formed using an aldehyde derivative of methoxy polyethyleneglycol. In this case, polyethylene glycol itself may be used as a side chain. Also it's derivatives having an hydroxy group protected by a group, not particularly limited but such as methoxy group or alkylthioethylene sulfonyl group, and having another hydroxy group being replaced by another group, not particularly limited but such as an amino group, a succinyl group, a carboxyl group, a carboxymethyl group, a thiol group, or dimethoxytrityl group may be used as a side chain. The molecular weight of the feasible polyethylene glycol or its derivative is not restricted, but it is preferably 300 to 20,000, or more preferably 1,000 to 8,000 as the optimal average molecular weight.

The proportion of the hydrophilic group to the carrier for nucleic acid stabilization is not restricted. It can be selected according to the type of the hydrophilic group and the purpose of use of the carrier. Normally, the proportion of the hydrophilic group to the carrier for nucleic acid stabilization is 10 to 95% by weight, preferably 20 to 95% by weight, more preferably 30 to 95% by weight, of the carrier. If the proportion is less than this range, a sufficient effect of solubilization based mainly on the hydrophilic group cannot be obtained. If the proportion is more than this range, sufficient cationic properties cannot be retained, and the stabilization based on compaction with DNA is not achieved.

Embodiments of the Novel Carrier for Nucleic Acid Stabilization Related to the Present Invention

The carrier for nucleic acid stabilization related to the present invention mainly exhibits the following effects:

(1) Easily solubilizes a complex of a gene to be introduced into a cell with the carrier;

(2) Suppresses the structural change of an exogenous gene and has the excellent function to retain the reversibility of the exogenous gene, and

(3) Stabilizes a DNA double helix or a DNA triple helix formed by introducing an exogenous gene into a cell.

The size and kind of a nucleic acid that can be introduced into a cell by the inventive carrier for nucleic acid stabilization are not particularly limited. Examples include linear double-stranded DNA, circular double-stranded DNA, oligonucleotide, and RNA. The use of the inventive carrier also makes it possible to introduce a structural gene encoding a protein useful for a cell, and to express this gene. For instance, an antisense can be introduced to control the expression of a particular gene. The inventive carrier for nucleic acid stabilization is also usable as a carrier for ribozyme, triplex or aptamer. Preferably used as the oligonucleotide are phosphodiesters, phosphorothioates, or other derivatives.

The amount of the inventive carrier for nucleic acid stabilization is not particularly limited, and is easy to optimize according to the purpose of use. For instance, it is preferred to use about 0.1 to 1,000 mol of the carrier relative to 1 mol of a nucleic acid.

The charge ratio to nucleic acid in complex formation using the inventive carrier for nucleic acid stabilization (i.e., the ratio of the number of the cationic amino acids of the carrier to the number of the phosphate groups of the nucleic acid, or [cationic amino acid groups of carrier]/[phosphate groups of nucleic acid]) can be optimized according to the purpose of use. Actually, with the carrier for nucleic acid stabilization, when the proportion of the hydrophilic group incorporated into the cationic poly(amino acid) is low, say 20 mol % or less, showing the compaction of the complex with DNA at the charge ratio of around 1. With the cationic homopolymer such as polylysine, the complex with DNA undergoes not only compaction, but also precipitation at the charge ratio of about 1. In the case of the complex of DNA with the carrier of the present invention, on the other hand, compaction occurs, but precipitation does not take place because of the hydrophilic groups of the carrier molecules. For example, the complex exists stably in a solution as colloidal particles. With the inventive carrier for nucleic acid stabilization, the preferred range for the charge ratio is 0.5 to 2, more preferably, 0.5 to 1.5. Outside these ranges, stabilization based on sufficient compaction will fail.

The carrier claimed in the present invention can also be used favorably in gene therapy based on autotransplantation which involves taking a target cell out of a patient s body, introducing a desired gene into this cell, and then returning this cell to the patient s body again (ex vivo gene therapy); as well as in gene therapy for directly administering a gene to a patient (in vivo gene therapy). The carrier can also be used preferably in both of a method which adds a new (normal) gene while leaving an abnormal (causative) gene intact (augmentation gene therapy), and a method of replacing an abnormal gene by a normal gene (replacement gene therapy).

The method of administering a preparation using the inventive carrier for nucleic acid stabilization is not particularly limited. The preparation may be administered parenterally, or preferably by injection. The amount of this carrier used differs depending on the method or purpose of use. When the carrier containing a nucleic acid is used by injection, for instance, it is preferred to administer it in a daily dose of about 0.1 μg/kg to 100 mg/kg, more preferably, about 1 μg/kg to 50 mg/kg.

The present invention will now be described in detail with reference to the following Examples, which do not limit the invention.

The present invention will be more fully illustrated with reference to the following non-limiting examples.

### Synthesis Examples 1 to 14

### Synthesis of Polymer By Modification With Dextran

### Synthesis Examples 1 and 2

Two poly-L-lysine solutions were prepared by dissolving 50 mg (0.24 mmol) of poly-L-lysine (PLL HBr, Mn=5.1×10⁴, Sigma) in 2 ml of dimethyl sulfoxide. A first dextran solution was obtained by dissolving 300 mg (5.17×10² mmol) of dextran (Dextran T10, Mn=5.8×10³, Pharmacia) in 3 ml of dimethyl sulfoxide (Kanto Chemical) (Synthesis Example 1). Similarly, a second dextran solution was obtained by dissolving 450 mg (7.75×10² mmol) of dextran in 4.5 ml of dimethyl sulfoxide (Synthesis Example 2). The first and second dextran solutions, respectively, were added to the poly-L-lysine solutions to prepare mixed solutions having dextran incorporation rates of about 20 mol % and about 30 mol %, respectively, on a charge basis. To each mixed solution, 30 μl of acetic acid (Kanto Chemical) was added. With stirring, 15 mg of sodium borocyanohydride (NaBH₃CN, Aldrich Chemical) was added to the 20 mol % mixed solution, while 2.5 mg of sodium borocyanohydride was added to the 30 mol % mixed solution, whereupon the poly-L-lysine and the dextran in the solution were graft reacted. After stirring for 2 days at 40° C., the system was purified with a dialysis membrane (molecular weight cutoff—10,000, Spectrum), and the product was lyophilized.

The amount of the dextran modified polymer yielded with a dextran incorporation rate of 20 mol % was 0.2314 g (yield 69%), while the amount yield for the polymer with a dextran incorporation rate of 30 mol % was 0.3576 g (yield 74%).

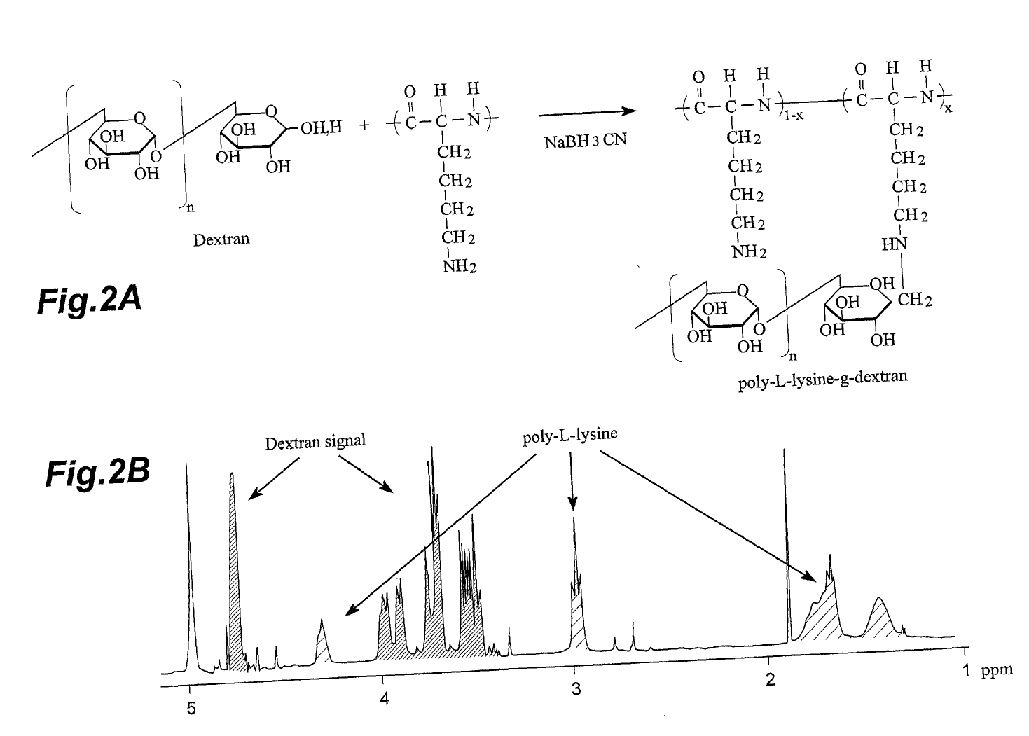

Whether the products were single substances or not was confirmed by gel permeation chromatography (GPC), which made sure that they had a purity of 95% or more. Confirmation of the dextran incorporation rates and the structures was performed by NMR (FIG. 2A and FIG. 2B), GPC and molecular weight analysis based on static light scattering.

### Synthesis Examples 3-14

In Synthesis Examples 3 to 14, synthesis was carried out under the same conditions as in Synthesis Examples 1 and 2, except that the conditions (number averaged molecular weights and amounts of charge of dextran, and amounts of charge of NaBH₃CN) shown in Tables 1 and 2 were employed.

TABLE 1
PLL-g-Dex(borate buffer. pH8.5)

| | | | | Copolymer | | | | | |
|---|---|---|---|---|---|---|---|---|---|
| | Reactants | | | | Molecular weight | | | Dextran Content | |
| Synthesis | PLL-HBr(*) | Dextran | NaBH₃CN | Yield | \overline{M}n/ | \overline{M}w/ | \overline{M}w/ | | % |

| Example | mg | mmol | Mn | mg | mmol | wt % | mg | mmol | % | 10000 | 10000 | Mn | wt % | graft |
|---|---|---|---|---|---|---|---|---|---|---|---|---|---|---|
| 1 | 50 | 0.2404 | 5900 | 300 | 0.0508 | — | 12.0 | 0.239 | 69 | — | — | — | — | — |
| 2 | 50 | 0.2404 | 5900 | 450 | 0.0763 | — | 22.5 | 0.358 | 74 | — | — | — | — | — |
| 3 | 150 | 0.721 | 2600 | 94 | 0.036 | 50.57 | 22.7 | 0.361 | 26.88 | 8.2 | 12.0 | 1.46 | 43.7 | 3.8 |
| 4 | 150 | 0.721 | 2600 | 187 | 0.072 | 67.08 | 45.0 | 0.716 | 78.64 | 9.5 | 12.8 | 1.35 | 60.3 | 7.5 |
| 5 | 150 | 0.721 | 2600 | 375 | 0.144 | 80.32 | 91.0 | 1.448 | 66.35 | 11.2 | 18.9 | 1.69 | 74.3 | 14.2 |
| 6 | 150 | 0.721 | 2600 | 937 | 0.360 | 91.07 | 227.0 | 3.612 | 70.51 | 22.7 | 30.6 | 1.35 | 87.3 | 34.0 |
| 7 | 150 | 0.721 | 5900 | 212 | 0.036 | 69.77 | 22.7 | 0.361 | 62.29 | 8.3 | 13.1 | 1.58 | 66.0 | 3.6 |
| 8 | 150 | 0.721 | 5900 | 425 | 0.072 | 82.23 | 45.0 | 0.716 | 40.56 | 13.5 | 19.2 | 1.42 | 79.3 | 7.1 |
| 9 | 150 | 0.721 | 5900 | 850 | 0.144 | 90.25 | 91.0 | 1.448 | 70.51 | 30.5 | 36.2 | 1.19 | 88.3 | 14.0 |

*\overline{M}w = 7700
Synthesis Examples 1and2 used PLL-HBr(Sigma) \overline{M}w51,000.
—Data not yet obtained.

TABLE 2
PLL-g-Dex Synthesis

| | | | | saccharide content |
|---|---|---|---|---|
| | | | | In copolymer |
| Synthesis | PLL-HBr(51k) | Dex(5900) | NaBH₃CN | % |

| Example | mg | mmol | mol % | mg | mmol | mmol | mg | graft | wt % |
|---|---|---|---|---|---|---|---|---|---|
| 10 | 100 | 0.48 | 1.77 | 50 | 8.47E − 03 | 3.98E − 02 | 2.5 | 1.77 | 33.33 |
| 11 | 100 | 0.48 | 3.53 | 100 | 1.69E − 02 | 7.96E − 02 | 5 | 3.53 | 50.00 |
| 12 | 100 | 0.48 | 5.30 | 150 | 2.54E − 02 | 1.19E − 01 | 7.5 | 4.70 | 69.47 |
| 13 | 100 | 0.48 | 10.59 | 300 | 5.08E − 02 | 2.39E − 01 | 15 | 9.58 | 83.00 |
| 14 | 100 | 0.48 | 21.19 | 600 | 1.02E − 01 | 4.77E − 01 | 30 | 19.73 | 91.89 |

Synthesis Example 10-14 used PLL-HBr(Sigma) Mw46,000

### Synthesis Examples 15 to 17

### Synthesis of PLL-g-PEG

400 mg of Poly-L-lysine (molecular weight 5,000 to 10,000) was dissolved in 4 ml of dimethyl sulfoxide in each of three different flasks. To the solutions, 2, 4 and 8 ml, respectively, of a dimethyl sulfoxide solution (230 mg/ml) of an aldehyde derivative (PEG-Aldehyde, molecular weight 5,000, Shearwater) of methoxyethylene glycol were added to make mixtures. Sodium borocyanohydride was further added in amounts of 35, 70 and 140 mg, respectively, and dissolved. Then, 30 μl of acetic acid was added to each flask, and the reaction was performed for 7 days at 40° C. After completion of the reaction, each reaction mixture was subjected to dialysis and ultrafiltration to isolate the resulting polymers (Table 3).

TABLE 3
PLL-g-PEG

| | Reactants | | | | | | Copolymer | | | | | |
|---|---|---|---|---|---|---|---|---|---|---|---|---|
| | | | Methoxy-PEG-aldehyde | | | | | Moleculan weight | | | PEG content | |
| Synthesis | PLL-HBr | | \overline{M}n/ | | | | Yield | \overline{M}n/ | \overline{M}w/ | \overline{M}w/ | wt % | % |
| Example | mg | mmol | 1000 | mg | mmol | mol % | % | 1000 | 1000 | \overline{M}n | (NMR) | graft |
| 15 | 400 | 53 | 5 | 469.5 | 93.9 | 5 | 9.7 | 40 | 44 | 1.1 | 65.2 | 4.8 |
| 16 | 400 | 53 | 5 | 939 | 187.8 | 10 | 7.5 | 57.9 | 66 | 1.1 | 76.4 | 8.3 |
| 17 | 400 | 53 | 5 | 1878 | 375.6 | 20 | 11.3 | 68.5 | 79.7 | 1.2 | 85.4 | 15 |

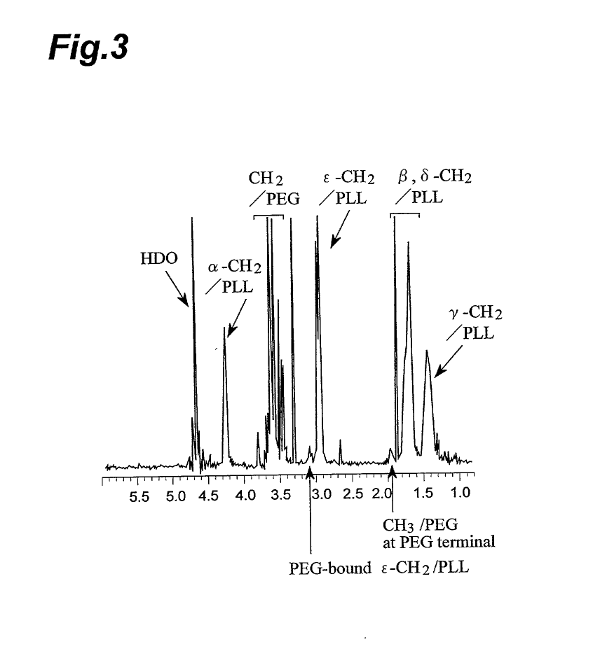

Table 3 shows the results, including the results obtained under other conditions for synthesis. The typical spectra of ¹H-NMR of the resulting polymers in heavy water are shown in FIG. 3. The following apparatus was used for ¹H-NMR measurement in the following description: (Varian, UNITY400Plus Spectrometer).

### EXAMPLE 1

### Formation of Complex of Dextran Modified Polymer with DNA

The turbidity or precipitation of an ionic complex of DNA with the poly-L-lysine-dextran modified polymer (PLL-g-Dex) having a dextran incorporation rate of 20 mol % (90 wt. %), obtained by synthesis in the Synthesis Example 14, was spectroscopically evaluated based on the absorbance of a solution measured at 500 nm.

To 1 ml of a solution of DNA (calf thymus, Sigma) adjusted to a concentration of 100 μg/ml (0.303 μmol/ml) with PBS or a 1:100 dilution of PBS, a solution of the dextran modified polymer was added at a varying charge ratio to make the total volume 2.5 ml. Here, the charge ratio means the ratio of the number of mols of the amino groups of the polymer to the number of mols of the phosphate groups of DNA. The turbidity of the complex in PBS and PBSx1/100 with a low ionic strength was measured.

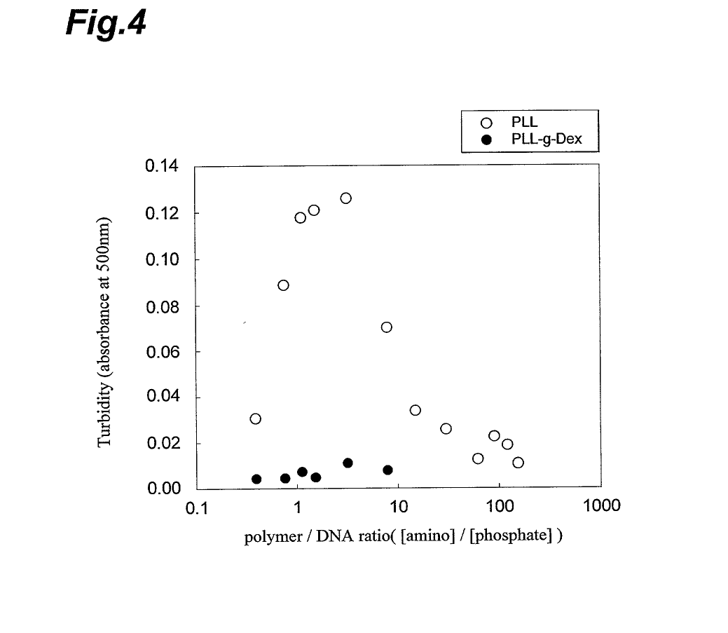

The results obtained on the solubility in PBS of the complex of DNA with the dextran modified polymer with a dextran incorporation rate of 20 mol % are shown in FIG. 4 along with the results on the solubility of a complex of polylysine with DNA. The numbers on the ordinate indicate the turbidity as absorbance at 500 nm, while the numbers on the abscissa indicate the polymer/DNA charge ratio. Measurements with the dextran comb-linked copolymer were made at the charge ratio ranging from 0 to 8. The dextran modified polymer, when DNA was added in an equimolar amount or even in an excess amount, showed no precipitation, proving high solubility. This is because the complex formed by the mixing of DNA and the polymer became soluble upon the incorporation of the hydrophilic dextran chain.

### Comparative Example 1

As a comparative example, the turbidity or precipitate formation of an ionic complex of DNA with poly-L-lysine (PLL.HBr, Peptide Research Institute) was investigated by the same method as in Example 1 using a solution of the poly-L-lysine. The charge ratio for the poly-L-lysine was varied from 0 to up to 158.

When the polylysine and DNA were mixed at a charge ratio of about 1, compaction occurred, and precipitation took place at a physiological ionic strength. At PBSx1/100, no precipitation was observed, while precipitation occurred in the presence of the excess polylysine or DNA, although this data is not shown.

### EXAMPLE 2

### Measurement of Circular Dichroism (CD)

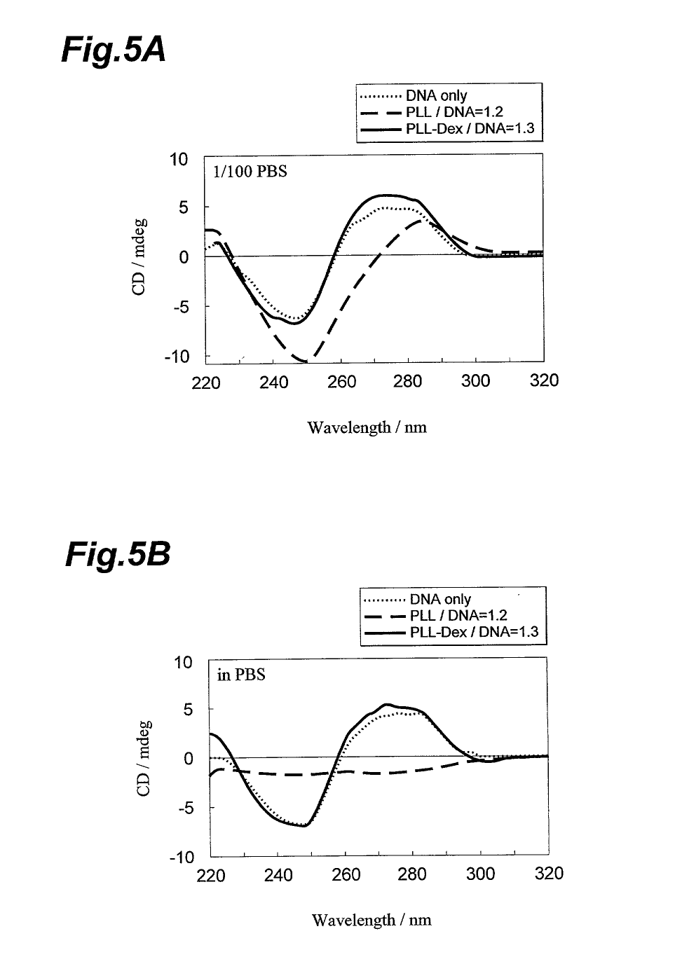

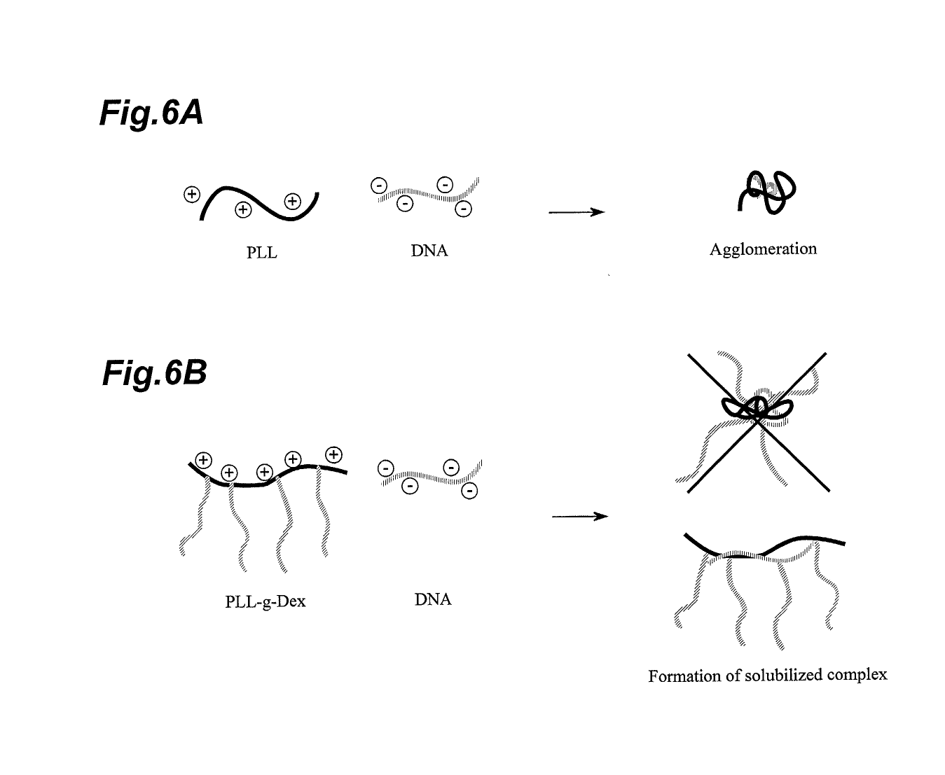

The structural change of DNA in the complex of DNA with the dextran modified polymer related to the present invention was evaluated by the CD spectrum. The circular dichroism (CD) was measured with a JASCO J-600 spectropolarimeter. Measurements all used the sample employed for turbidimetry (the dextran modified polymer synthesized by Synthesis Example 1). The results on the CD spectrum are shown in FIGS. 5A and 5B, which indicates changes in the CD spectra of the complex of the dextran modified polymer with DNA in 1/100 PBS and the complex of the dextran modified polymer with DNA in PBS. The CD spectrum showed little change even after the addition of the dextran modified polymer. This held true at a physiological ionic strength. That is, DNA interacts with the dextran modified polymer only weakly, and thus its structure does not change greatly. The complex of the polymer with DNA, as indicated schematically in FIGS. 6A and 6B, does not cause marked change to the structure of DNA, but solubilizes DNA because of interaction. As will be described later on, DNA is not decomposed by enzymes in the serum, although the DNA is bound without a marked change in its structure. This fact demonstrates that the incorporation rate of dextran is as high as 20 mol % (a dextran chain is incorporated into one of the five amino groups of polylysine); therefore, the potent interaction between polylysine and DNA can be suppressed, and the structure of DNA can be maintained. Furthermore, the dextran group suppresses the degradation of DNA by an enzyme reaction. These results reveal that the polymer of the present invention binds to DNA, and forms a complex in which the repulsion between the anions in the double or triple helix of the DNA is suppressed by the cations of polylysine, the double or triple helix is stabilized, and further the structure of the DNA is maintained. Thus, the complex enables a single-stranded DNA to recognize other single strand or a double helix, promoting the formation of a double or triple helix.

### Comparative Example 2

As a comparative example, the CD spectrum of the structural change of DNA in a DNA-poly-L-lysine complex was evaluated by CD spectrum under same condition of Example 2. The results are shown in FIGS. 5A and 5B, which indicates changes in the CD spectrum of the complex of poly-L-lysine with DNA in 1/100 PBS. In PBS, the complex of poly-L-lysine with DNA forms a precipitate, and thus its CD spectrum was measured in a 1:100 dilution of PBS where no precipitation occurs. The CD spectrum for DNA alone was given as a dotted line. This DNA is B DNA, because it shows positive values at 260 to 300 nm, and negative values at 220 to 260 nm. Upon addition of poly-L-lysine to this DNA, the curve shifts to the right, changing the CD spectrum greatly, with the negative values in the range 220 to 260 nm becoming larger, and the positive values in the range 260 to 300 nm becoming smaller. This finding shows that DNA changed from type B to a type similar to type C (Dexter S. Moore and Thomas E. Wagner, Biopolymers, Vol. 13, 977-986 (1974)). That is, the complex of polylysine with DNA undergoes a marked structural change as a result of compaction. As shown schematically in FIGS. 6A and 6B, the polylysine-DNA complex is compacted, and precipitated under physiological ionic conditions.

### EXAMPLE 3

### Stabilizing Effect of Dextran Comb-linked nd Copolymer

The stabilizing effect of the dextran modified polymer of the present invention on double-stranded DNA was examined based on the melting curve of DNA.

The dextran modified polymer and DNA (poly(dA) and poly(dT), each homopolymer with about 200 bases, Pharmacia) were formed into a complex in PBS (10 mM, pH=7.2, 0-150 mM NaCl, 0.5 mM EDTA, phosphate buffer) with the charge ratio being varied progressively, 0, 0.2, 0.5, 1, 2 and 10. The DNA concentrations in these solutions were set at 0.4 units/ml. The solution of the complex was heated up to 110° C. at a rate of 0.2° C./min or 0.5° C./min, and then its temperature was lowered. The absorbance at 260 nm during this period were measured with a spectrophotometer (DU Series 600, Beckmann).

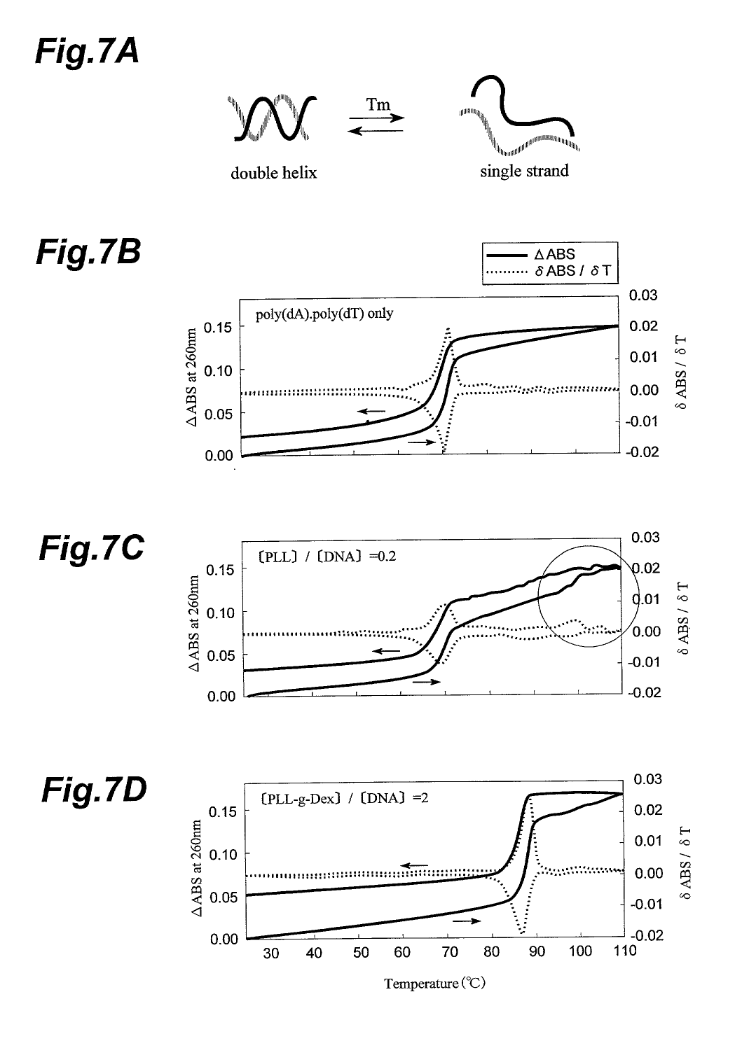

Similar measurements were made on the complex of the dextran modified polymer with DNA with the NaCl concentration in PBS being varied as 0 mM, 50 mM, 150 mM and 1,000 mM. FIGS. 7A and 7B shows the results of the measurements made on the double-stranded DNA comprising poly(dA) and poly(dT). The solid lines indicate changes in the absorbance with changes in the temperature, while the dotted lines indicate the values of the corresponding primary differentials. The data in the drawings of FIGS. 7A and 7B were all obtained in PBS with 150 mM NaCl. It is general knowledge that as the temperature of a double-stranded DNA solution is increased, the complementary strands of the double helix separate from each other at a certain temperature or higher, forming random coils. This denaturation increases the UV absorption by about 40%. This is called a hyperchromic effect, which is said to occur for the lack of interactions between the electrons of the bases proximal to each other. This DNA denaturation is a cooperative phenomenon, in which partial breakage of the structure leads to instability of the remaining part of the structure. Thus, the hyperchromic effect at 260 nm occurs in a very narrow range of temperature. Curves showing the above changes were taken as the melting curves of DNA, in which the transition point was chosen as the melting point Tm that was determined by the primary differentials.

The Tm for the double helix of DNA can be interpreted as stable, but varies with the solvent, the type and concentration of the ions in the solution, and pH. The Tm is also more stable in a G-C base pair with three hydrogen bonds than in an A-T base pair with two hydrogen bonds. Thus, the Tm rises as the mole fraction of the G-C base pairs increases. To help confirm the stabilizing effect of the inventive polymer, therefore, poly(da) and poly(dT) stable and having a low melting point was used.

As the denatured DNA solution is gradually cooled down below Tm, the double helix revives. This is known as DNA annealing. The temperature optimal for annealing is known to be a temperature about 25° C. lower than Tm (Voet Biochemistry (Part 2), Donald Voet, translated by Tamiya et al., Tokyo Kagaku Dojin). In FIGS. 6A and 6B, the melting point of DNA alone was 72° C. in PBS in the presence of NaCl. When the temperature was lowered, rewinding of a double helix was observed. The lowermost chart gives the results for a solution containing the dextran modified polymer added at a charge ratio 2 to DNA. The melting point Tm was noted at 89 ° C. Since a rewinding was observed unlike polylysine-DNA as a control, the polymer of the present invention was found not to inhibit the formation of a double helix from single strands. These results demonstrate that polylysine greatly stabilizes a double helix, and that a dextran group incorporated into polylysine permits the reconstitution of a double helix from single strands, a phenomenon unobserved with polylysine to be described later on, although such dextran weakens the stabilizing effect slightly.

### Comparative Example 3

As a control, the stabilizing effect of polylysine on a double-stranded DNA was examined based on the melting curve of DNA in the same manner as in Example 3.

Poly-L-lysine (PLL-HBr, Peptide Research Institute) and DNA (poly(dA) and poly(dT), each homopolymer with about 200 bases, Pharmacia) were formed into a complex in PBS (10 mM, pH=7.2, 0-150 mM NaCl, 0.5 mM EDTA, phosphate buffer) so that there would be excess DNA to a degree to which no precipitation would occur. The melting curve in the middle chart of FIGS. 7A-7D gives the results obtained when polylysine was added at a charge ratio of 0.2. The transition at a temperature about 70 ° C. represents the transition of only DNA not interacting with polylysine. As shown in the differential curve at 100° C. on the higher temperature side (the encircled region), the melting point of the polylysine-DNA complex was observed. Polylysine markedly stabilized a double helix, but when the temperature was lowered, no rewinding occurred. Single-stranded DNA s were compacted with polylysine, and became unable to form a double helix. A similar phenomenon was noted in the melting curve of a double helix, although not shown.

### EXAMPLE 4

### Stabilizing Effect of the Inventive Polymer on DNA Triple Helix

A DNA triple helix was formed from poly(dA) and poly(dT) in an amount of twice the number of mols of poly(dA). The stabilizing effect of the combed copolymer on the triple helix was investigated based on a melting curve in the same manner as described above.

The exact concentrations of poly(dA) (Pharmacia) and poly(dT) (Pharmacia) were determined by molar extinction coefficient. The molar extinction coefficient of poly(da) at 257 nm was 8,900. The molecular extinction coefficient of poly(dT) at 265 nm was 9,000. Poly(dA) and poly(dT) were placed in PBS at a poly(dA):poly(dT) molar ratio of 1:2 using the precisely measured molarity. Then, the dextran modified polymer of the present invention was added at a charge ratio to DNA of 0, 0.2, 0.5, 1, 2 and then 10. Each of the solutions was held at 90° C. for 10 minutes, and then allowed to stand overnight at room temperature.

The temperature of the solution was raised to 110° C. at a rate of 0.2° C./min or 0.5° C./min, and then lowered. During this period, the absorbance was measured (Thresia Thomas and T. J. Thomas, Biochemistry, Vol. 32, No. 50, 14068-14074 (1993)). The same measurements as for the double helix were made with the NaCl concentration in PBS being varied as 0 mM, 50 mM, 150 mM and 1,000 mM. For comparison with the stabilizing effect of the polymer on the triple helix, measurements were made on spermine (Wako Pure Chemical), a low molecular polyamine, with the charge ratio ([amino groups of spermine]/[phosphate groups of DNA]) being varied similarly.

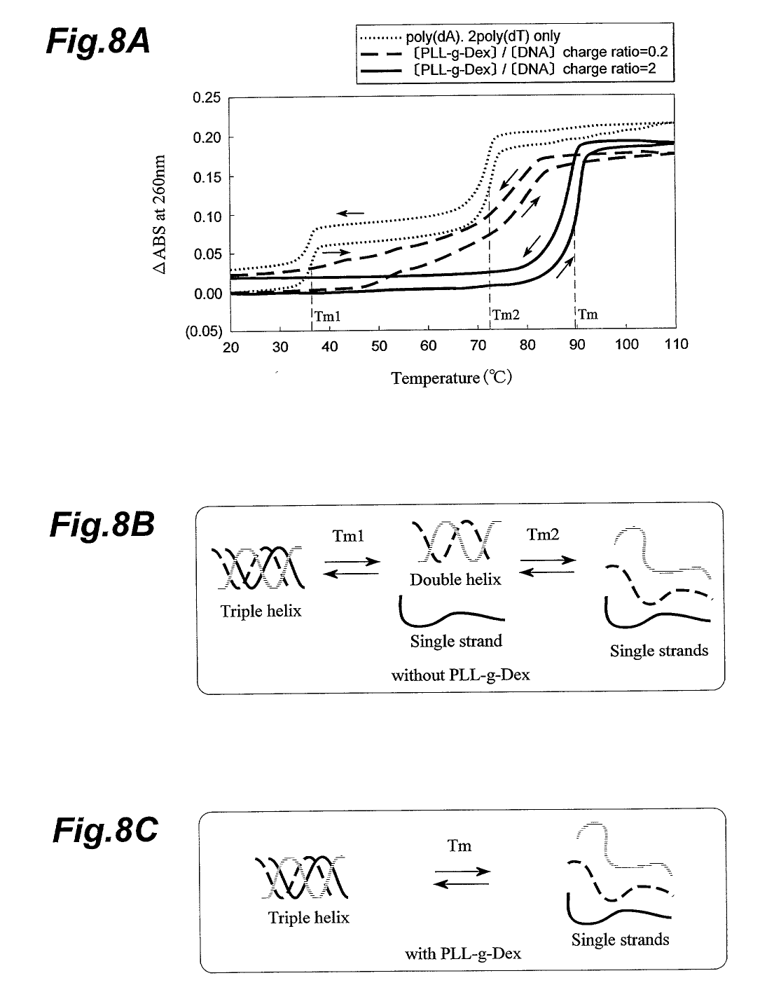

FIGS. 8A and 8B shows the melting curves of the DNA triple helix. The dotted line indicates the curve for the DNA alone. With the DNA alone, transition points were obtained at 37° C. and 73° C. This finding shows that as the temperature increases, transition takes place from the triple helix to a double helix, and then to single strands. When the charge ratio of the inventive polymer to the DNA was changed to 0.2 and then to 2, the transition position shifted to the higher temperature side. With the addition of the dextran modified polymer at a charge ratio of 2, a transition point was observed at 89° C. The change in the absorbance at this time was nearly equal to the change in the absorbance of the DNA alone. This shows that at the melting point Tm, the inventive polymer causes the triple helix to be converted into single strands at a stroke. These results show that the addition of the inventive polymer stabilizes the double helix and triple helix of DNA while maintaining their reversibility. Particularly for the DNA triple helix, the stabilizing effect is high in view of the fact that the melting point is raised by more than 50° C.

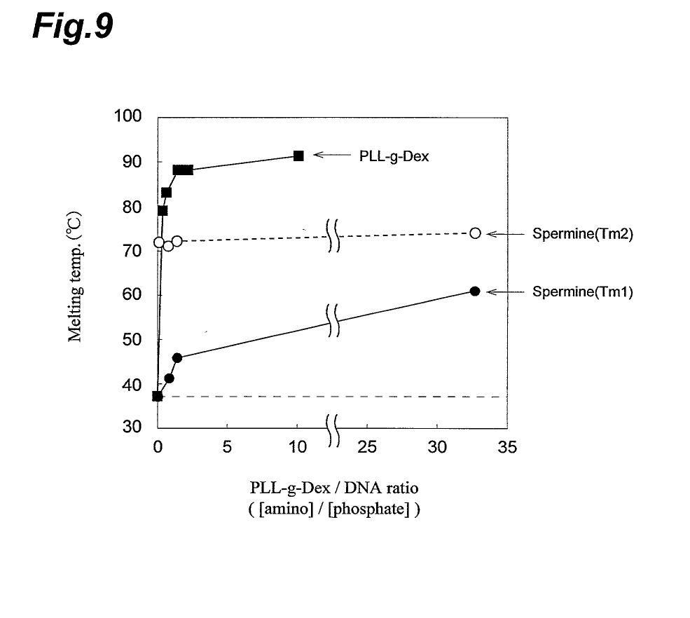

FIG. 9 shows the results of the dependency of the melting point of the triple helix, Tm, on the concentration of the dextran modified polymer. The ordinates indicate the melting point, and the abscissas indicate the charge ratio of the combed copolymer to DNA. The control used here was spermine said to be the strongest stabilizer of a triple helix of low molecular polyamines (Thresia Thomas and T. J. Thomas, Biochemistry, Vol. 32, No. 50, 14068-14074 (1993)). Spermine gives two melting points, Tm1 and Tm2. The melting point Tm1 of the triple helix is found to go up by about 20° C. at a spermine/DNA charge ratio of 33. With the dextran modified polymer of the present invention, on the other hand, the melting point is shown to rise by as high as 50° C. at a charge ratio of only 1 to 2, indicating an excellent stabilizing effect compared with spermine. This may have been because the modified polymer of the invention is a polyvalent polymeric cation.

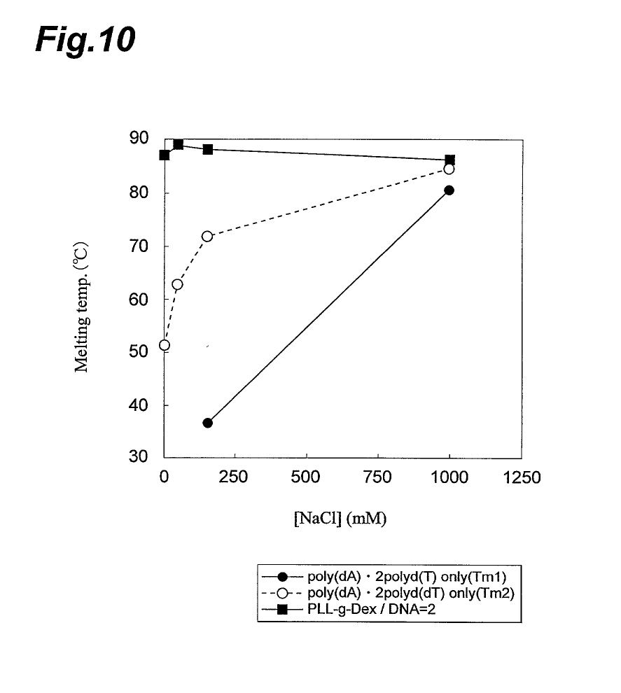

FIG. 10 shows the results of the dependency of the melting point of a DNA triple helix on the concentration of NaCl. The ordinates indicate the melting point, and the abscissas indicate the concentration of NaCl in PBS. A combination of the dextran modified polymer with DNA at a charge ratio of 2 was compared with DNA alone. With DNA alone, two melting points exist, Tm1 and Tm2, as in FIGS. 15A-15C. Below an NaCl concentration of 150 mM, no Tm1 is present, thus showing that no triple helix is formed. That is, because of many ions present, repulsion between the anions of DNA weakens, showing the stabilization of the double or triple helix of DNA. Based on measurements at lowering temperatures, the rewinding of duplex or triplex occurs at a high ionic strength, but minimally occurs at a low ionic strength. That is, ions such as NaCl promote and stabilize the double or triple helix formation of DNA (Thresia Thomas and T. J. Thomas, Biochemistry, Vol. 32, No. 50, 14068-14074 (1993)).

The dextran modified polymer gives one melting point of a double or triple helix (FIG. 9), 89° C. Unlike DNA alone, rewinding of a double or triple helix was observed at a low ionic strength (not shown). Even at a high NaCl content, the complex was found to be highly stabilized. These results prove that the polymer of the present invention is an excellent carrier for in vivo stabilization which promotes and markedly stabilizes DNA double or triple helix formation, regardless of changes in the ionic strength.

### Comparative Example 4

A DNA triple helix was formed from poly(dA) and poly(dT) in an amount of twice the number of mols of poly(da) in the same manner as in Example 4. The stabilizing effect on the triple helix was similarly investigated based on a melting curve.

For comparison with the stabilizing effect of the inventive polymer on the triple helix, measurements were made on spermine (Wako Pure Chemical), a low molecular polyamine, with the charge ratio ([amino groups of spermine]/[phosphate groups of DNA]) being varied similarly.

FIG. 9 shows the results of the dependency of the melting point of the triple helix, Tm, on the charge ratio. The ordinates indicate the melting point, and the abscissas indicate the charge ratio. The control used here was spermine said to be the strongest stabilizer of a triple helix of low molecular polyamines (Thresia Thomas and T. J. Thomas, Biochemistry, Vol. 32, No. 50, 14068-14074 (1993)). Spermine gives two melting points, Tm1 and Tm2. The melting point Tm1 of the triple helix is found to go up by about 20° C. at a spermine/DNA charge ratio of 33.

### EXAMPLE 5

### Electrophoresis

The action of the complex of DNA and the inventive carrier for nucleic acid stabilization to suppress the decomposition of DNA by serum enzymes was confirmed by experiments described below. That is, the stability in the serum of the complex of DNA with the polymer of the present invention was examined by electrophoresis.

25 μl of FCS (fetal calf serum, Gibco) was added to 500 ng/0.4 μl of plasmid DNA, and the mixture was incubated at 37° C. for 1, 2 and 6 hours to prepare samples as samples after decomposition of plasmid DNA.

A mixture containing the dextran modified polymer of the present invention added to plasmid DNA in the serum at a charge ratio of 1 was also prepared.

Furthermore, a complex of the same amount of plasmid DNA with the dextran modified polymer incorporated at a charge ratio of 1 to plasmid DNA was formed in PBS. 25 μl of FCS was added, and the mixture was incubated at 37° C. for 1, 2 and 6 hours to prepare samples. The concentration of FCS was 97%.

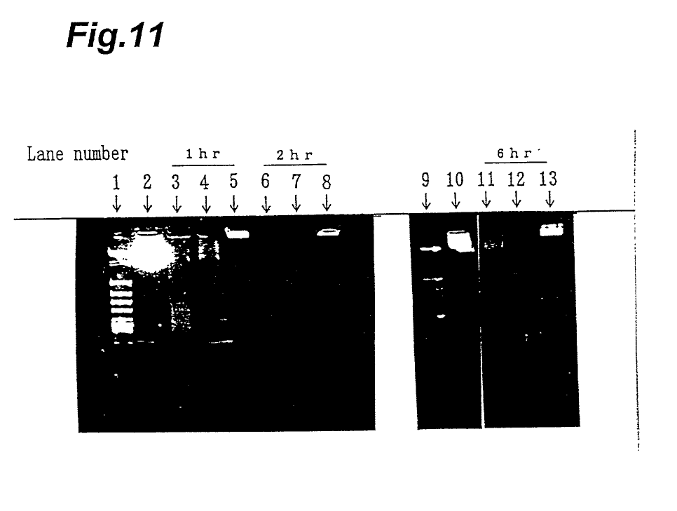

The prepared samples were electrophoresed on 2% agarose gel (containing ethidium bromide). FIG. 11 shows the results of electrophoresis for examining the stability of the complex of DNA with the carrier of the present invention in the serum. Lanes 1 and 9 represent markers, while lanes 2 and 10 represent the untreated plasmid DNA. Lane 3 represents the plasmid DNA that was incubated at 37° C. for 1 hour in the serum for decomposition by serum enzymes. Lane 4 represents the plasmid DNA that was similarly incubated at 37° C. for 1 hour in the serum, but containing the dextran modified polymer added at a charge ratio of 1. Lane 5 represents the plasmid DNA that was formed into the complex with the dextran modified polymer incorporated at a charge ratio of 1, and then similarly incubated in the serum for 1 hours at 37° C. Lanes 6, 7 and 8 represent the preparations treated in the same manner except that the conditions, 37° C. and 1 hour, were replaced by 37° C. and 2 hours. Lanes 11, 12 and 13 give similar testing results involving the incubation time of 6 hours. In lanes 3 and 6, the bands detected in the untreated plasmid DNA disappeared, and bands were detected on the lower molecular weight side. These findings point to the decomposition of plasmid DNA by serum enzymes. In lanes 4 and 7, the bands were lost to bands detected on a slightly lower molecular weight side. Since the detected bands were at a slightly upper location, unlike lanes 3 and 6, small DNA resulting from decomposition forms some complex with the polymer of the present invention, achieving an increased molecular weight. Lanes 5, 8 and 13 showed immobile bands. Since no bands were detected at any other locations, unlike lanes 3, 4, 6 and 7, it is shown that the DNA in the complex of the inventive polymer with the DNA is stable to enzymes in the serum, and is not decomposed thereby. The appearance of the bands on the high molecular weight side relative to the untreated plasmid DNA shows that the undecomposed plasmid DNA and the dextran modified polymer forms a complex, attaining an increased molecular weight.

### EXAMPLE 6

### Complex of DNA With Dextran Modified Polymer With Low Dextran Content

In Example 1, when poly-L-lysine was formed into a complex with DNA at a charge ratio of 1, precipitation occurred, increasing turbidity. A complex of DNA with a dextran modified polymer with a high dextran content (20 mol %) caused no precipitation, and did not become turbid (see FIG. 4).

The complex of DNA with the dextran modified polymer with a low dextran content was measured for the absorbance (at 260 nm) and turbidity (absorbance at 340 nm) at various charge ratios.

The dextran modified polymer used was one which contained dextran having a molecular weight of 2.6 k (called Dex2600) or 5.9 k (called Dex5900) graft polymerized with poly-L-lysine, and whose dextran content ranged from 4 to 34 mol %. The buffer solution used was that of the composition 10 mM PBS, 150 mM NaCl and 0.5 mM EDTA (pH 7.2).

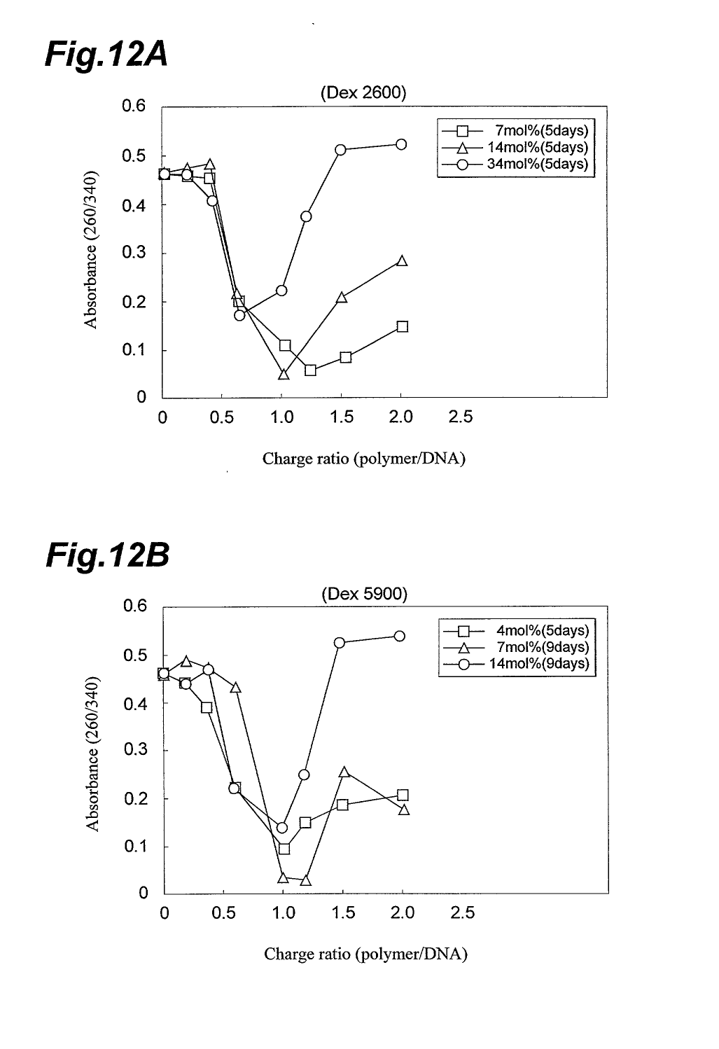

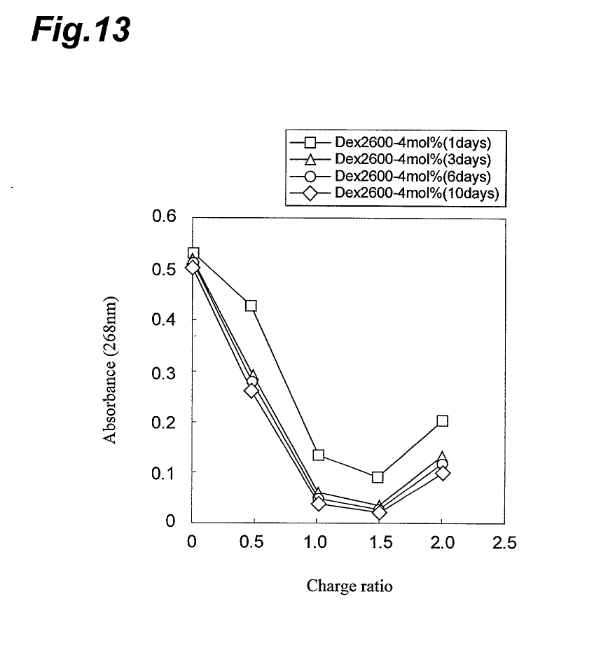

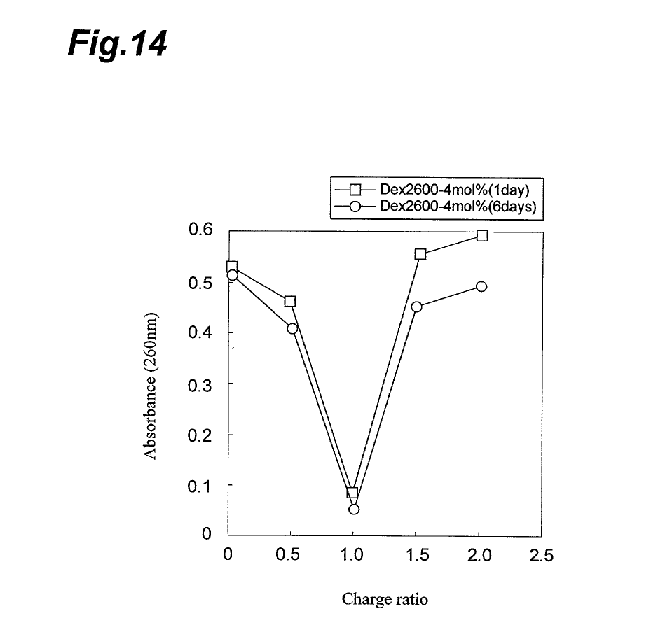

FIGS. 12A and 12B gives the results obtained when poly(dA) and poly(dT), each homopolymer with about 200 bases, were used as DNA. FIG. 13 gives the results obtained when oligonucleotide (30 mer synthetic random oligonucleotide, Nippon Seifun, Flour Mills) was used as DNA. FIG. 14 also shows the results obtained when plasmid (pSV (6.8 k), Promega) was used.

As shown in FIGS. 12A and 12B, the absorbance commonly decreased at a charge ratio of around 1. At a charge ratio of 1, no turbidity was confirmed in any samples with a naked eye, nor was turbidity shown based on the absorption at 340 nm. These results held true of the use of the oligonucleotide and plasmid.

The reason why the absorbance at 260 nm based on DNA decreases at a charge ratio of around 1 may be that the polylysine portion of the dextran modified polymer and DNA undergo compaction. On the other hand, the dextran modified polymer-DNA complex does not cause agglomeration or precipitation as observed with the polylysine-DNA complex, but can exist stably (be dispersed) in water. This may be because the dextran portion of the polymer is present on the surface of the compaction product.

As described in the foregoing, the carrier for nucleic acid stabilization of the present invention has the following properties:

(1) Easily solubilizes a complex of a gene to be introduced into a cell with the carrier;

(2) Suppresses the structural change of an exogenous gene and has the excellent function to retain the reversibility of the exogenous gene, and

(3) Has the effect of stabilizing a DNA double helix or a DNA triple helix formed by introducing an exogenous gene into a cell.

Thus, the carrier for nucleic acid stabilization of the present invention is useful as an in vivo transporter of an oligonucleotide gene therapeutic agent for various genetic diseases or viral diseases such as AIDS. Particularly, it is useful as an in vivo transporter of a gene therapeutic agent involving formation of a DNA triple helix.

The inventive carrier also contributes greatly to the creation of various useful animals and plants by genetic engineering technologies, including virus-resistant plants.

From the invention thus described, it will be obvious that the invention may be varied in many ways. Such variations are not to be regarded as a departure from the spirit and scope of the invention, and all such modifications as would be obvious to one skilled in the art are intended for inclusion within the scope of the following claims.

What is claimed is:

1. A carrier for stabilizing a nucleic acid, said carrier having a poly(cationic amino acid) as a main chain, and having as a side chain a hydrophilic group chemically bound to the poly(cationic amino acid) in a combed shape.

2. The carrier of claim 1, wherein the poly(cationic amino acid) is polylysine.

3. The carrier of claim 1, wherein the hydrophilic group is at least one member selected from the group consisting of polyethylene glycol, polyethylene glycol derivatives, and saccharides.

4. The carrier of claim 1, wherein the hydrophilic group is at least one member selected from the group consisting of glycosaminoglycan, dextran, polyethylene glycol, and polyethylene glycol derivatives.

5. The carrier of claim 1, wherein the poly(cationic amino acid) and the hydrophilic group are graft-polymerized.

6. The carrier of claim 1, wherein 10 to 95% by weight of the carrier for nucleic acid stabilization is the hydrophilic group.

7. The carrier of claim 1, wherein the ratio of X to Y (X/Y) is in the range 0.5 to 2, X being the number of the cationic amino acid groups constituting the poly(cationic amino acid), and Y being the number of the phosphate groups contained in the DNA to be formed into a complex with the carrier for nucleic acid stabilization.

8. A method for stabilizing a nucleic acid forming a complex of the nucleic acid with a carrier for stabilizing a nucleic acid, said carrier having a poly(cationic amino acid) as a main chain, and having as a side chain a hydrophilic group chemically bound to the poly(cationic amino acid) in a combed shape.

9. The method of claim 8, wherein the ratio of X to Y (X/Y) is in the range 0.5 to 2, X being the number of the cationic amino acid groups constituting the poly(cationic amino acid), and Y being the number of the phosphate groups contained in the DNA to be formed into a complex with the carrier for nucleic acid stabilization.
